(19) United States
(12) Patent Application Publication
(10) Pub. No.: US 2013/0131419 A1
(43) Pub. Date: May 23, 2013

(54) GIBBSITE CATALYTIC CRACKING CATALYST

(71) Applicants: John Scott Buchanan, Lambertville, NJ (US); William A. Wachter, Flemington, NJ (US); Kun Wang, Bridgewater, NJ (US); Kathryn L. Peretti, Clinton, NJ (US); Daniel Mark Giaquinta, Saratoga, CA (US); Hongyi Hou, San Jose, CA (US)

(72) Inventors: John Scott Buchanan, Lambertville, NJ (US); William A. Wachter, Flemington, NJ (US); Kun Wang, Bridgewater, NJ (US); Kathryn L. Peretti, Clinton, NJ (US); Daniel Mark Giaquinta, Saratoga, CA (US); Hongyi Hou, San Jose, CA (US)

(73) Assignee: EXXONMOBIL RESEARCH AND ENGINEERING COMPANY, Annandale, NJ (US)

(21) Appl. No.: 13/302,443

(22) Filed: Nov. 22, 2011

(51) Int. Cl. C07C 4/00; C10G 11/04; B01J 29/80; B01J 29/04; B01J 29/08

(52) U.S. Cl. 585/800; 502/64; 502/65; 502/67; 502/68; 208/120.01

(57) ABSTRACT

A fluid catalytic cracking catalyst exhibiting reduced coke make comprises a zeolite cracking component in a matrix of gibbsite having a median particle size of not more than 0.4 microns and preferably not more than 0.3 microns. The zeolite cracking component will normally be a faujasite, with preference to zeolite Y in its various forms such as Y, HY, REY, REHY, USY, REUSY and secondary zeolite additives may be present, including ZSM-5.

### FIELD OF THE INVENTION

This invention relates to mesoporous catalytic cracking catalysts. More particularly, catalytic cracking catalysts formulated with gibbsite and rare earth oxides show improved coke selectivity.

### BACKGROUND OF THE INVENTION

Fluid catalytic cracking (FCC) is a well-established and widely used process for converting high boiling hydrocarbon feedstocks to lower boiling, more valuable products. In the FCC process, the high boiling feedstock, typically a vacuum gas oil (VGO) is contacted with a cracking catalyst in the substantial absence of hydrogen at elevated temperatures. The cracking reaction typically occurs in the riser portion of the catalytic cracking reactor with the catalyst particles entering near the bottom of the riser near the feed injection nozzles. The catalyst is a small particle size material with a particle size that permits it to be fluidized in the unit. Cracked hydrocarbon products are separated from catalyst by means of cyclones and coked catalyst particles are stripped with steam and sent to a regenerator where coke is burned off the catalyst. The regenerated catalyst is then recycled to contact more high boiling feed at the beginning of the riser.

Typical FCC catalysts contain active crystalline aluminosilicates, principally the faujasite zeolites (particularly zeolite Y in its various forms such as REY, HY, REHY, USY, REUSY) with ZSM-5 frequently present to improve gasoline product octane or improve olefin yield. The zeolite(s) as the active cracking component and any active inorganic oxide matrix components will be composited in the particles with a binder generally formed from an amorphous gel or sols such as silica sol which acts to bind the components together on drying; the binder may or may not have activity. Fillers such as clays of the kaolin type make up the balance of the catalyst composition. The oxide matrix materials should be attrition resistant, selective with regard to the production of hydrocarbons without excessive coke make and not readily deactivated by metals. Matrices with cracking activity (active matrices) may assist in the overall cracking reaction. Current FCC catalysts may contain in excess of 40 wt % zeolites. At these high zeolite concentrations, it is difficult to maintain a pore structure that is highly mesoporous while at the same time highly active and selective.

U.S. Pat. No. 5,221,648 describes a FCC catalyst which is a composite of a crystalline aluminosilicate zeolite within a mesoporous silica-alumina matrix. The matrix has a polymodal pore size distribution and is attrition-resistant and selective in the production of olefins. U.S. Pat. No. 4,908,405 discloses a FCC process employing a catalyst composition comprised of a monodispersed mesoporous aluminosilicate matrix material having pore diameter between about 100 and 500 Å, alumina and a crystalline zeolite. U.S. Pat. No. 4,010,116 is directed to zeolite catalysts having improved thermal stability. The catalysts incorporate a synthetic mica-montmorillonite aluminosilicate, in admixture with a pseudoboehmite, AlO(OH)H₂O. The pseudoboehmite may contain crystalline aluminum trihydroxides, Al(OH)₃, such as bayerite and gibbsite. Upon calcination at 500° C., pseudoboehmite converts to gamma alumina. Therefore, a fresh catalyst initially containing pseudoboehmite will contain increasing amounts of gamma alumina as it ages in the FCC unit and passes successively through the regenerator. Both pseudoboehmite and gibbsite can convert to various phases of alumina (e.g. chi, gamma, kappa, theta, and eventually alpha) upon heating. The aluminas that are formed in the catalyst from these precursors are believed to be especially effective in cracking the heavier components of the FCC feed which cannot penetrate into the pores of the zeolite component of a typical FCC catalyst. From this point of view, it would be desirable to use gibbsite in the inorganic matrix of a FCC catalyst because of its enhanced cracking activity for the heavy ends and, in addition, it is abundant and inexpensive. However, gibbsite is known to have a low surface area and is relatively inert in terms of its activity and has therefore been little used in FCC catalysts.

U.S. Pat. No. 5,961,817 describes FCC catalysts made with 5-50% aluminum trihydroxide (mainly gibbsite) matrices and with silica sol binders. Examples show a catalyst with gibbsite showing lower specific coke (and lower activity) in the microactivity test (MAT) than comparable pseudoboehmite catalyst made with silica sol binders. U.S. Pat. No. 6,022,471 describes FCC catalysts similar to those in U.S. Pat. No. 5,961,817 except that rare earth metal is added by post-exchange of the rare earth onto the formulated, spray-dried catalyst. The gibbsite-containing catalysts develop significant pores of 160 to 320 Å.

The milling of gibbsite can affect its subsequent phase transformations upon heating: the transition to alpha-alumina is subject to mechanical activation in this way, as described by S. W. Jang, et al in J. Mater. Sci. Lett. 19 (2000) 507-510 and further in J. Ceram. Proc. Res. 2[2] (2001) 67-71; Jang's findings demonstrate that milling of gibbsite affected the phases into which it transforms for a given time and temperature of heating.

### SUMMARY OF THE INVENTION

We have now determined that gibbsite can be expected to follow different phase trajectories from boehmite as it is heated in an FCC unit. This can lead to different catalytic performance for the different precursor materials (i.e., gibbsite versus boehmite). Furthermore, different forms, in particular, different particle sizes, for a given precursor have been discovered to affect the phase trajectory of that material and its catalytic performance: in particular, the use of small particle size gibbsite has been found to result in a significant reduction in the coke yield at constant conversion and bottoms yield.

According to the present invention, a fluid catalytic cracking catalyst comprises a zeolite cracking component in a matrix of gibbsite having a median particle size of not more than 0.4 microns, preferably not more than 0.3 microns, and most preferably not more than 0.2 microns. The zeolite cracking component will normally be a faujasite, with preference to zeolite Y in its various forms such as Y, HY, REY, REHY, USY, REUSY.

The catalyst will be used in a fluid catalytic cracking process in which a high boiling, heavy oil cracking feed is contacted with hot, fluidized catalyst at an elevated cracking temperature in the cracking zone of the unit, typically at the foot of the riser cracking zone, to crack the heavy oil feed into lighter boiling hydrocarbons with these cracking reactions being carried out in the absence of added hydrogen (although some hydrogen may be released during the cracking). The cracked hydrocarbons are separated from catalyst after leaving the cracking zone usually by means of cyclones or other types of disengaging devices and the coked catalyst particles are stripped with steam and sent to a regenerator where coke is burned off the catalyst. The regenerated catalyst, having gained heat from the combustion of the coke is then recycled to contact more high boiling feed in the cracking zone at the foot of the riser.

### FIGURES

FIG. 1 is a graph plotting the effect of gibbsite median particle size on cracking coke yield relative to feed conversion.

FIG. 2 is a graph plotting the effect of gibbsite median particle size on cracking coke yield relative to bottoms yield.

### DETAILED DESCRIPTION

The catalyst will contain one or more zeolites as the principle cracking component. A wide variety of crystalline aluminosilicate zeolites, both natural and synthetic, having catalytic cracking activity are known, commercially available and can be used in the practice of this invention. One zeolite will be a large pore size zeolite; zeolites preferred as the main cracking component are the faujasites, more especially are zeolite Y and those isostructural with zeolite Y, including Y, REY, HY, REHY, USY and REUSY. Secondary or additive zeolite components may be present, normally, the medium pore size zeolites, especially ZSM-5, for various purposes, especially improving gasoline octane or increasing olefin yield. The faujasite zeolite component is typically included in the catalyst in an amount of about 5 to about 50 wt %, preferably about 20 to about 40 wt %, and preferably the large pore zeolite or the whole catalyst may have rare earth added to it. The addition of rare earth to the faujasite is preferred without rare earth added to the matrix, e.g., as by impregnation onto the entire catalyst. Thus, in the present catalysts, the gibbsite matrix will preferably be free of rare earths. If an additive zeolite such as ZSM-5 is present, it will normally be used in amounts from about 5 to about 25 wt %, depending on the feed and purpose of the additive zeolite.

The matrix material of the present catalysts is gibbsite of small median particle size. It constitutes a mesoporous matrix having pore diameters in the range between about 100 to about 300 Å, in order to permit access to the interior pore structure by the feed. In addition to the zeolite component or components and matrix material, the catalyst particles will additionally comprise a binder such as a gel or sol (e.g. silica sol and a filler), typically a naturally occurring, relatively non-porous clay conventionally used for the purpose such as kaolinite, bentonite, hectorite, sepiolite, attapulgite, montrnorillonite, halloysite. The preferred hinders for the present catalysts are the alumina-rich binders such as aluminum chlorhydrol (ACH, Al₂Cl(OH)₅) or peptized alumina, i.e. analyzing at least 40 wt % alumina, preferably at least 50 or 60 wt % alumina. The catalyst is preferably in the form of microspheres formed in the spray drying process with the size of the spheres being initially in the range of about 10 to about 200 microns, more preferably from about 60 to about 100 microns although attrition will tend to reduce the size as the catalyst ages in use and progressively degrades.

In a preferred process for the preparation procedure of the catalysts herein, the zeolite(s), gibbsite matrix, binder and, preferably, one or more clay an filler materials are added together or in sequence, in any order, and slurried at ambient temperature with water. In general, the weight ratio of water:solids in the slurry can range between about 1.5:1 to about 4:1, preferably between about 1.7:1 to about 2:1. When the weight ratio of water:solids is less than about 1.4, the viscosity of the slurry may be too high for spray drying, and when the weight ratio of water:solids exceeds about 4:1 the attrition-resistance of the catalyst tends to be poor. The clay filler component is added to, or slurried with the zeolite, matrix and the binder. The pH of the slurry preferably ranges between about 4 and about 10; the addition of the clay to the slurry does not normally alter the pH of the slurry significantly or at all. A typical catalyst of invention may comprises about 20 to 35 wt % of an REY faujasite as the cracking component, about 10 to 25 wt % of gibbsite, about 5 to 15% aluminum chlorhydrol as binder, and about 35 to 50 wt % as clay filler.

Gibbsite has the chemical formula Al(OH)₃ and is the principal constituent of bauxites. It has a monoclinic crystal symmetry with four molecules per unit cell. It is commercially available from Alcoa™ in particles sizes ranging from 0.3 to 2.0 microns under the tradename SPACERITE™. A median particle size of less than 0.4 microns is preferred for the gibbsite component, with a more preferred of value less than 0.3 microns, and yet more preferred of less than 0.2 microns, with particle sizes measured typically with a SediGraph™ instrument, using ASTM method C958. A preferred proportion of gibbsite in the final catalyst is about 10 to about 40 solids wt % with about 15 to about 25% more preferred. It is preferred to have rare earth present in the zeolite, preferably added by ion exchange. This may be done at various points in the catalyst manufacturing process, including rare earth exchange before the zeolite is added to the whole catalyst, or rare earth exchange of the final catalyst microspheres.

In conducting the catalytic cracking operation the conditions used, apart from the use of the present gibbsite bound catalysts, will be generally typical of an the process with feedstocks typically consist mainly of vacuum gasoil or atmospheric resids, but may include other refinery streams or biologically-derived feedstocks such as vegetable oils, pyrolysis oils, and materials derived from algae. The cracking temperature (i.e., typically the riser top temperature) preferably ranges from 500° C. to about 700° C., more preferably from about 510° C. to about 600° C., with a pressure ranging from about 100 kPa to about 1100 kPa, preferably from about 200 kPa to about 400 kPa. Suitably, catalyst/oil ratios in the cracking zone for use with the catalysts herein are not more than about 30:1, and may range from about 2:1 to about 10:1, more usually from about 4:1 to about 9:1. Suitable regeneration temperatures include a temperature ranging from about 1100 to about 1500° F. (600 to about 800° C. at a pressure ranging from 100 to about 1100 kPa.

### ADDITIONAL EMBODIMENTS

Additional embodiments of the invention herein are as follows.

Embodiment 1. A fluid catalytic cracking catalyst which comprises a zeolite cracking component in a gibbsite matrix having a median particle size of not more than 0.4 microns.

Embodiment 2. The catalyst of embodiment 1, wherein the median particle size of gibbsite is not more than 0.3 microns.

Embodiment 3. The catalyst of embodiment 1, wherein the median particle size of gibbsite is not more than 0.2 microns.

Embodiment 4. The catalyst of any of the above embodiments, wherein the gibbsite matrix is substantially free of rare earth.

Embodiment 5. The catalyst of any of the above embodiments, wherein the zeolite cracking component comprises a large pore size faujasite.

Embodiment 6. The catalyst of embodiment 5, wherein the large pore size faujasite comprises a rare earth.

Embodiment 7. The catalyst of any of the above embodiments, wherein the zeolite cracking component comprises zeolite USY.

Embodiment 8. The catalyst of any of the above embodiments, wherein the catalyst is further comprised of aluminum chlorhydrol as a binder.

Embodiment 9. The catalyst of any of the above embodiments, wherein the matrix is a mesoporous matrix having pore diameters in the range between about 100 to 300 Å.

Embodiment 10. The catalyst of any of the above embodiments, which comprises ZSM-5 as a secondary zeolitic cracking component.

Embodiment 11. The catalyst of embodiment 10, which comprises from about 5 to about 25 wt % of ZSM-5 as a secondary zeolitic cracking component.

Embodiment 12. The catalyst of any of the above embodiments, which comprises from about 5 to about 50 wt % of the zeolite cracking component and from about 5 to about 50 wt % of the gibbsite matrix.

Embodiment 13. The process according to any of embodiments 1-11, an which comprises from about 20 to about 40 wt % of the zeolite cracking component and from about 10 to about 60 wt % of the gibbsite matrix.

Embodiment 14. The process according to any of embodiments 1-11, which comprises from about 20 to about 35 wt % of an REY faujasite as the cracking component, about 10 to about 25 wt % of the gibbsite matrix, about 5 to about 15 wt % aluminum chlorhydrol as binder, and about 35 to about 50 wt % clay filler.

Embodiment 15. A fluid catalytic cracking process which comprises contacting a high boiling, heavy oil cracking feed with particles of a hot, fluidized catalyst comprising a zeolite cracking component in a gibbsite matrix having a median particle size of not more than 0.4 microns at an elevated cracking temperature in the cracking zone to crack the heavy oil feed into lighter boiling hydrocarbons in the absence of added hydrogen and form coke on the catalyst, separating the cracked hydrocarbons from coked catalyst in a disengaging zone, stripping the coked catalyst particles, regenerating the stripped coked catalyst by burning the coke off the catalyst and recycling hot, regenerated catalyst to contact more high boiling feed in the cracking zone.

Embodiment 16. The process of embodiment 15, wherein the median particle size of gibbsite in the cracking catalyst is not more than 0.3 microns.

Embodiment 17. The process of embodiment 15, wherein the median particle size of gibbsite in the cracking catalyst is not more than 0.2 microns.

Embodiment 18. The process according to any of embodiments 15-17, wherein the feed is cracked in the cracking zone at a cracking temperature from about 500° C. to about 700° C. at a catalyst:oil ratio of about 2:1 to about 10:1.

Embodiment 19. The process according of embodiment 18, wherein the feed is cracked in the cracking zone at a cracking temperature from about 510° C. to about 600° C., at a catalyst:oil ratio of about 4:1 to about 9:1.

Embodiment 20. The process according to any of embodiments 15-19, wherein the gibbsite matrix is substantially free of rare earth.

Embodiment 21. The process according to any of embodiments 15-20, wherein the zeolite cracking component comprises a large pore size faujasite.

Embodiment 22. The process of embodiment 21, wherein the large pore size faujasite comprises a rare earth.

### Example

A set of three catalysts was prepared and tested. These catalysts contained 25 wt % of a REY faujasite as the principle cracking component, 20 wt % of gibbsite, 10 wt % aluminum chlorhydrol as binder, with the balance (45 wt %) kaolin clay.

The three catalysts in this set differed in the median particle size of the gibbsite. Particle sizes of the gibbsites were measured with a SediGraph™ instrument (Model III), using ASTM Method C958. This instrument measures settling velocities of particles in an aqueous suspension. Particle sizes are calculated using Stokes' Law. A particle density of 2.42 g/cm3 was used in these calculations.

Catalyst A used gibbsite as received from the manufacturer. Catalyst B used gibbsite that had been milled in a laboratory ball mill. Catalyst C used gibbsite that was milled more severely. The median particle diameters of the three gibbsites used in these three catalysts were:

TABLE 1

| | Median particle diameter |
|---|---|
| | (microns) |
| Catalyst A | 1.14 |
| Catalyst B | 0.35 |
| Catalyst C | 0.17 |

Median particle diameter, microns

| | |
|---|---|
| Catalyst A | 1.14 |
| Catalyst B | 0.35 |
| Catalyst C | 0.17 |

After steaming for 16 hours at 778° C. to mimic refinery deactivation, these catalysts were tested in a laboratory catalytic cracking unit. In this unit, feed and catalyst traversed a reactor tube, to mimic the co-flow pattern in a refinery FCC unit. The teed was a vacuum gasoil-based refinery FCC feedstock.

The steamed catalysts had the following microporous and mesoporous surface areas, as measured by nitrogen physisorption:

TABLE 2

| | Microporous Surface | Mesoporous Surface |
|---|---|---|
| | Area | Area |
| | (m²/g) | (m²/g) |
| Catalyst A | 101 | 61 |
| Catalyst B | 97 | 59 |
| Catalyst C | 91 | 60 |

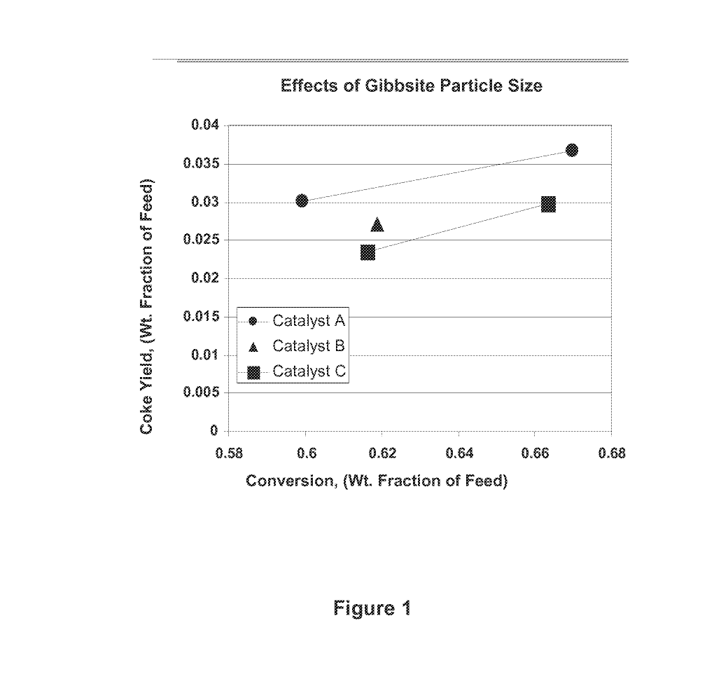

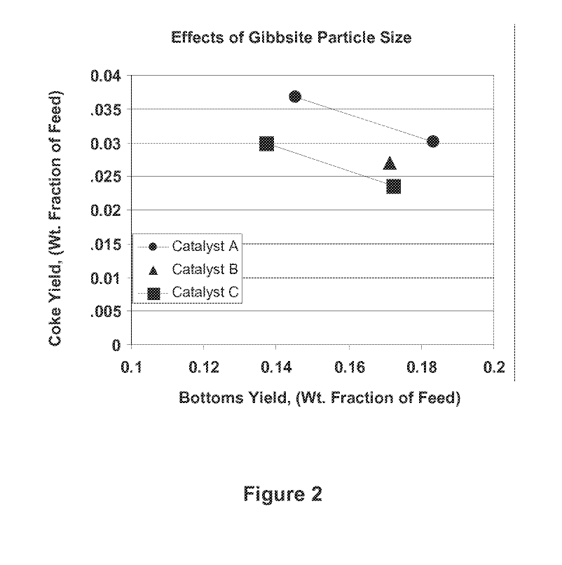

Key results from the cracking reactor are shown in FIGS. 1 and 2 which illustrate two different measures of the coke selectivity of the catalysts. The gibbsite with the smaller median particle size gives lower (better) coke selectivity, whether plotted versus conversion or bottoms yield. Here, “conversion” denotes conversion to coke plus material with a normal boiling point less than 220° C. (430° F.), “Bottoms” denotes material with a normal boiling point above 345° C. (650° F.).

The decreased coke yield and increased bottoms production has the potential for an improvement in the economics of the operation: even a modest increase in yields of valuable products can give a large economic benefit given the enormous scale of current units and their numbers in use. Many units are coke-limited (conversion cannot be increased without creating excessive and unacceptable coke formation on the catalyst), and bottoms is the least valuable fluid product, so the decrease in coke make is a key performance parameter for FCC catalysts. It indicates that use of the present catalysts can allow higher production of valuable products like olefins, gasoline, and distillate, without a concomitant increase in the production of coke.

What is claimed is:

1. A fluid catalytic cracking catalyst which comprises a zeolite cracking component in a gibbsite matrix having a median particle size of not more than 0.4 microns.

2. A fluid catalytic cracking catalyst according to claim 1, wherein the median particle size of gibbsite is not more than 0.3 microns.

3. A fluid catalytic cracking catalyst according to claim 2, wherein the median particle size of gibbsite is not more than 0.2 microns.

4. A fluid catalytic cracking catalyst according to claim 1, wherein the gibbsite matrix is substantially free of rare earth.

5. A fluid catalytic cracking catalyst according to claim 1, wherein the zeolite cracking component comprises a large pore size faujasite.

6. A fluid catalytic cracking catalyst according to claim 5, wherein the large pore size faujasite comprises a rare earth.

7. A fluid catalytic cracking catalyst according to claim 5, wherein the zeolite cracking component comprises zeolite USY.

8. A fluid catalytic cracking catalyst according to claim 1, wherein the catalyst is further comprised of aluminum chlorhydrol as a binder.

9. A fluid catalytic cracking catalyst according to claim 1, wherein the matrix is a mesoporous matrix having pore diameters in the range between about 100 to 300 Å.

10. A fluid catalytic cracking catalyst according to claim 1, which comprises ZSM-5 as a secondary zeolitic cracking component.

11. A fluid catalytic cracking catalyst according to claim 10, which comprises from about 5 to about 25 wt % of ZSM-5 as a secondary zeolitic cracking component.

12. A fluid catalytic cracking catalyst according to claim 1, which comprises from about 5 to about 50 wt % of the zeolite cracking component and from about 5 to about 50 wt % of the gibbsite matrix.

13. A fluid catalytic cracking catalyst according to claim 1, which comprises from about 20 to about 40 wt % of the zeolite cracking component and from about 10 to about 60 wt % of the gibbsite matrix.

14. A fluid catalytic cracking catalyst according to claim 1, which comprises from about 20 to about 35 wt % of an REY faujasite as the cracking component, about 10 to about 25 wt % of the gibbsite matrix, about 5 to about 15 wt % aluminum chlorhydrol as binder, and about 35 to about 50 wt % clay filler.

15. A fluid catalytic cracking process which comprises contacting a high boiling, heavy oil cracking feed with particles of a hot, fluidized catalyst comprising a zeolite cracking component in a gibbsite matrix having a median particle size of not more than 0.4 microns at an elevated cracking temperature in the cracking zone to crack the heavy oil feed into lighter boiling hydrocarbons in the absence of added hydrogen and form coke on the catalyst, separating the cracked hydrocarbons from coked catalyst in a disengaging zone, stripping the coked catalyst particles, regenerating the stripped coked catalyst by burning the coke off the catalyst and recycling hot, regenerated catalyst to contact more high boiling feed in the cracking zone.

16. A fluid catalytic cracking process according to claim 15, wherein the median particle size of gibbsite in the cracking catalyst is not more than 0.3 microns.

17. A fluid catalytic cracking process according to claim 16, wherein the median particle size of gibbsite in the cracking catalyst is not more than 0.2 microns.

18. A fluid catalytic cracking process according to claim 15, wherein the feed is cracked in the cracking zone at a cracking temperature from about 500° C. to about 700° C., at a catalyst:oil ratio of about 2:1 to about 10:1.

19. A fluid catalytic cracking process according to claim 18, wherein the feed is cracked in the cracking zone at a cracking temperature from about 510° C. to about 600° C., at a catalyst:oil ratio of about 4:1 to about 9:1.

20. A fluid catalytic cracking process according to claim 18, wherein the gibbsite matrix is substantially free of rare earth.

21. A fluid catalytic cracking process according to claim 20, wherein the zeolite cracking component comprises a large pore size faujasite.

22. A fluid catalytic cracking process according to claim 21, wherein the large pore size faujasite comprises a rare earth.
